(19) United States
(12) Patent Application Publication
(10) Pub. No.: US 2003/0187074 A1
(43) Pub. Date: Oct. 2, 2003

(54) Oral compositions for treatment of diabetes

(76) Inventors: Javed Hussain, Aurangabad, IN (US); Parag Prakash Borde, Aurangabad, IN (US); Harshal Prabhakar Bhagwatwar, Aurangabad, IN (US); Manjusha Malhotra, New Delhi, IN (US); Milind C. Shukla, Aurangabad, IN (US); Noel John De Souza, Mumbai, IN (US)

Correspondence Address: KENYON & KENYON, ONE BROADWAY, NEW YORK, NY, 10004, US

(21) Appl. No.: 10/379,314

(22) Filed: Mar. 3, 2003

Related U.S. Application Data
(60) Provisional application No. 60/364,691, filed on Mar. 15, 2002.
(60) Provisional application No. 60/385,787, filed on Jun. 3, 2002.

(30) Foreign Application Priority Data
Mar. 4, 2002 (IN) 200/MUM/2002
Jun. 4, 2002 (IN) 498/MUM/2002

(51) Int. Cl. A61K 9/14; A61K 31/155

(52) U.S. Cl. 514/635; 424/488; 424/486

(57) ABSTRACT

An oral delivery system for the treatment of non-insulin dependent diabetes mellitus in humans includes a pharmaceutically effective amount of a biguanide and a water-insoluble polymeric carrier, providing for a controlled release of the biguanide independent of environmental pH. In addition, a layer including a glitazone or sulfonylurea can be included in the oral delivery system, while still providing for a pH-independent, controlled release of biguanide over an extended period of time.

### FIELD OF THE INVENTION

This invention describes a controlled release delivery system of a biguanide and its combinations with rosiglitazone or sulphonylureas for the treatment of non-insulin dependent diabetes mellitus (NIDDM) and in improving glycemic control. The invention also describes processes for the preparation of such a delivery system and its use in the treatment of diabetes.

### BACKGROUND OF THE INVENTION

Diabetes mellitus of type II is a progressive metabolic disorder with diverse pathologic manifestations and is often associated with lipid metabolism and glycometabolic disorders. The long-term effects of diabetes result from its vascular complications; the microvascular complications of retinopathy, neuropathy and nephropathy and the macrovascular complications of cardiovascular, cerebrovascular and peripheral vascular diseases. Initially, diet and exercise is the mainstay of treatment of type II diabetes. However, these are followed by administration of oral hypoglycemic agents. Current drugs used for managing type II diabetes and its precursor syndromes such as insulin resistance, include classes of compounds, such as, among others, biguanides, thiazolidinediones and sulfonylureas.

Biguanides, represented principally by metformin, phenformin and buformin, help in the control of blood glucose by inhibiting hepatic glucose production, reducing intestinal absorption of glucose and enhancing peripheral glucose uptake. Biguanides, especially metformin, lowers both basal and post-prandial plasma glucose and thus improves tolerance of glucose in patients. Metformin exerts normoglycemic action with reduced risk of lactic acidosis and is also known to lower blood triglyceride levels. It is therefore a preferred mode of therapy among biguanides.

Thiazolidinediones, represented principally by the class of glitazones including, for example, rosiglitazone, troglitazone and pioglitazone, among others, act by increasing the sensitivity of insulin receptors in the body and decreasing peripheral insulin resistance. Thiazolidinediones, preferably rosiglitazone, stimulate adipogenesis and reduce plasma triglyceride and free fatty acid concentrations. These enhance insulin action at the cellular level but do not stimulate insulin release, nor do they mimic its action.

Sulfonylureas, represented principally by glipizide, glimiperide, glyburide, glibornuride, glisoxepide, gliclazide acetohexamide, chlorpropamide, tolazamide, and tolbutamide, among others, help in controlling or managing NIDDM by stimulating the release of endogenous insulin from the beta cells of the pancreas.

Biguanides, thiazolidinediones and sulfonylureas are commercially available in the form of tablets of the individual drugs, either immediate release (IR) formulations or in some cases controlled release (CR) formulations, to be administered orally to patients in need thereof, in protocols calling for the single administration of the individual ingredient. Metformin monotherapy is used as a first line treatment in diabetic patients but may be supplemented with other drugs when the secondary failure of the therapy sets in. The addition of a thiazolidinedione agent to concurrent biguanide treatment provides a balance of stimulated release of insulin while ameliorating insulin resistance and thus provides a level of glycemic control unattainable by either medication alone. Similarly, the administration of biguanide with sulfonylurea provides optimum glycemic control where monotherapy of each is found to be inadequate. But, multiple medications such as these for the prophylaxis or treatment of diseases usually result in patient inconvenience and consequently, patient non-compliance to the prescribed dosage regimen. The ease of using combination therapy for multiple medications as opposed to separate administrations of the individual medications has long been recognized in the practice of medicine. Such a therapy provides therapeutic advantage for the benefit of the patient and the clinician. Further, such therapy provides both increased convenience and improved patient compliance resulting form the avoidance of missed doses through patient forgetfulness.

Oral combination products should ideally be adaptable so that release rates and profiles can be matched to physiological and chronotherapeutic specifications. To formulate an effective combination product, it is pertinent to consider physicochemical properties of the drugs and their behavior in the physiological environment of the gastrointestinal tract. The pH of the tract varies between 1.2 to more than 7.4 from stomach to colon. Further, variables such as the gastric residence time are subject to very significant interindividual variations and are inter alia dependent on the nutritional habits of the individual. However, upon oral administration, the drug formulation usually traverses the stomach in about 1-2 hours. This relatively short gastric residence time necessitates pH-independent release, from formulations, for drugs absorbed even beyond stomach, say, in the upper gastrointestinal tract. Also, it may require frequent dosing for drugs with short half-lives and brings forth the need for controlled release formulations. The pH-independent controlled release of such drugs has been a long sought objective. An example of such a drug, whose bioavailability is highly dependent on the local physiology of the gastrointestinal tract, is metformin.

Metformin is freely soluble in water (>300 mg/ml at 25° C.). It is absorbed extensively from the upper proximal region of the gastrointestinal tract and has poor absorption from the distal region. The absolute bioavailability of a 500 mg metformin hydrochloride tablet given under fasting conditions is approximately 50-60%. It shows a lack of dose proportionality with increasing doses due to decreased absorption indicating a saturable absorption process or permeability/transit time limited absorption. It has a plasma elimination half-life of about 3 hours that makes it a suitable candidate for extended release formulations. Development of a controlled release dosage form of this biguanide has thus been through approaches to retain the dosage form in the stomach or the proximal part of the small intestine. Such approaches as the use of floating devices, or mucoadhesive polymers to enhance adhesion to the gastrointestinal mucus, or systems which contain swellable polymers which swell in the stomach on contact with aqueous fluids, or the use of dosage forms of shapes which do not allow them to be emptied from the stomach are described in the prior art (WO 99/47128 to Timmins et al., U.S. Pat. No. 6,099,859 to Cheng et al., U.S. Pat. Nos. 6,284,275 B1 and 6,099,862 both to Chen et al., U.S. Pat. No. 6,120,803 to Wong et al., U.S. Pat. No. 20010018070 A1 to Shell et al., U.S. Pat. No. 5,955,106 to Moeckel et al.). All these applications adopt the principle of use of water-swellable polymers alone or along with other hydrophobic materials for enhancing the retention of the dosage form in the gastrointestinal tract. The expanded dosage forms may block the pyloric sphincter or may cause gastric discomfort following multiple dosing resulting from retention of swollen dosage units in the stomach. Further, these systems show variable release profiles based on environmental pH of the gastrointestinal tract. Also, some of the polymeric systems used show mucoadhesive properties. The mucoadhesion is a function of the amount of mucus present, the rate of turnover of the mucus and the strength of the interaction with the mucus, leading to release profile variations depending on these parameters. Floating based systems demonstrate variability in drug release as the retention in the stomach is a function of food contents, quantity, and the type of food, to name a few.

For the above stated reasons, the oral controlled drug delivery systems heretofore described are not completely satisfactory. An oral drug delivery composition providing a pH-independent, controlled release of metformin would alleviate the aforementioned problems associated with the swelling or floating type of delivery systems. A delivery system utilizing this concept is provided by the present invention.

Insulin resistance is a common feature characterizing the pathogenesis of type II diabetes. Metformin improves glucose tolerance but cannot enhance insulin sensitivity. Rosiglitazone, a member of the thiazolidinedione class of antidiabetic agents, improves glycemic control by improving insulin sensitivity. Rosiglitazone is a highly selective and potent agonist for the peroxisome proliferator-activated receptor-gamma (PPARγ). Activation of PPARγ nuclear receptors regulates the transcription of insulin responsive genes involved in the control of glucose production, transport, and utilization. In addition, PPARγ-responsive genes also participate in the regulation of fatty acid metabolism. The antidiabetic activity of rosiglitazone has been demonstrated in type II diabetes in which hyperglycemia and/or impaired glucose tolerance is a consequence of insulin resistance in target tissues. A single administration of rosiglitazone activates the insulin receptors for an extended period and may thus be administered as a single dose without there being a need to maintain the plasma concentration of this drug.

A combination therapy of a biguanide, in particular, metformin, and rosiglitazone has a synergistic effect on glucose control, since both agents act by different but complementary mechanisms. The method of treating diabetes by employing combinations of biguanides and glitazones has been demonstrated in clinical evaluation (see WO 00/27401 and U.S. Pat. No. 6,011,049). Pharmaceutical compositions having combinations of biguanides and thiazolidinediones providing controlled or immediate release of both of the drugs are known in the art. For example, U.S. Pat. No. 6,296,874 to Cutie et al. and published U.S. patent application Ser. Nos. 20010036478 A1, 2010034374 A1, and 20010046545 A1, all to Adjei et al., describe controlled release core combinations of a glitazone with a biguanide chosen from metformin, phenformin or buformin in a single dosage form. The patent applications to Adjei et al. discuss the preparation of such combinations using either silicate polymers or polysaccharides.

U.S. Pat. Nos. 6,166,043 and 6,172,090 claim a method for reducing the amounts and side effects of active components administered to a diabetic patient, which comprises administering a therapeutically effective amount of an insulin sensitivity enhancer in combination with a biguanide. The insulin sensitivity enhancer claimed in the system is a thiazolidinedione selected from pioglitazone and troglitazone, while the biguanide is selected from metformin, phenformin and buformin. The combination is administered as an admixture or the agents are administered independently. The thiazolidinedione and the biguanide are available after oral administration of the dosage form as a conventional immediate release composition.

Sulfonylureas lower blood glucose levels acutely by stimulating the release of insulin from the pancreas, an effect dependent upon functioning beta cells in the pancreatic islets. They bind to sulfonylurea receptors on the beta cell plasma membrane, causing closure of ATP-sensitive potassium channels leading to depolarization of the cell membrane. This in turn opens voltage-gated calcium channels, allowing influx of calcium ions and subsequent secretion of insulin.

The use of combinations of metformin (a biguanide) and glyburide (a sulfonylurea) has been demonstrated to be synergistic in clinical trials when compared with the use of the individual agents separately (see Physician's Desk Reference 2000, page 832). The monograph also advocates the use of combinations of metformin and sulfonylureas for patients not controlled on metformin alone. Several prior art references pertain to pharmaceutical compositions having combinations of biguanides and sulfonylureas providing for controlled or immediate release of both of the drugs. For example, a unit-dose combination of metformin and glipizide as an immediate release formulation is commercially available (Zidmin™ tablets, Wockhardt), and a combination dosage form of metformin and glyburide for immediate release is described in U.S. Pat. No. 6,303,146 to Bonhomme et al.

Extended release tablets, which employ either a biguanide drug alone or a sulfonylurea drug alone, have been described in the prior art. For example, WO 96/08243 discloses a controlled release dosage form containing only metformin hydrochloride as the active ingredient, and employs a hydrogel to push the active ingredient from the dosage form. Similarly, U.S. Pat. Nos. 5,545,413, 5,591,454 and 5,091,190 disclose controlled release dosage forms containing only the drug glipizide, and employ a hydrogel to push the active ingredient from the dosage form.

U.S. Pat. Nos. 6,099,862 and 6,284,275, both to Chen et al., describe a combination composition for the simultaneous controlled release of a biguanide and a sulfonylurea. The composition comprises a core containing the two active agents along with other excipients and a semipermeable controlled release coating from which the release of the active agents is controlled by the presence of at least one passageway in the coat. Though the composition claims to achieve a controlled release of both the active agents, the composition suffers from certain drawbacks. The formation of a passageway in the coating requires expensive equipment such as a laser-hole drilling machine or an accurate mechanical drill for drilling the hole in the coat. The formation of the holes in the coat could also be achieved through the use of pore formers added into the coating itself. However, the use of a coated tablet composition is associated with the possibility of dose dumping on coating failure resulting in toxicity to the patient. Both the coating process as well as the laser-hole drilling process are time consuming and require great care to be taken for a number of processing parameters including the spray rate, polymer concentration in the polymer solution, grade of the polymer, the percentage weight gain, the type and percentage of plasticizer or pore former used, the diameter of the drilled hole, and such other parameters related to the formation of the coating, in order to achieve reproducible results. The compositions described in these patents are dependent completely on the coat and its characteristics, such as its thickness and permeability, the presence or absence of plasticizer(s)/pore former(s)/laser hole(s), and such others, for the controlled release of the biologically active agents. Furthermore, these compositions release the biologically active agents immediately in the absence of the coating.

As is evident, several pharmaceutical compositions are described in the reference literature that relates to drug delivery systems. However, for the above stated reasons, and because the prior art discloses either complicated devices and systems which are difficult to manufacture on the industrial scale, or the components used therein are not user friendly, the oral drug delivery systems heretofore described are not completely satisfactory.

Furthermore, for the administration of a combination of a biguanide with a glitazone or sulfonylurea for synergistic effect in the treatment of NIDDM, the individual commercially available products have been heretofore administered together. There is no availability in clinical practice of such combinations of a pH-independent controlled release biguanide along with an immediate release glitazone or sulfonylurea, all in one physically and chemically stable dosage form for ready administration. The provision of pH-independent controlled release biguanide alone or in a fixed-dose combination with an immediate release glitazone or sulfonylurea would fill a highly desired gap in the medical armamentarium. Such a product would improve the treatment of NIDDM through significantly enhanced patient compliance because of ease of administration and a reduced frequency of dosing. There is also the possibility of a significant reduction in the doses of the drug substances used in combination because of the synergistic action, resulting in a possible reduction in toxicity.

Combinations of biologically active agents are especially difficult to formulate because of the inherent differences in physicochemical properties, the possible drug-drug interactions between the drugs and also in the ingredients used for formulation of the combination composition.

The compositions exemplified for controlled release of metformin in the prior art references contain very high amounts of polymers (hydrophilic) that upon contact with gastric fluids swell to form a soft gelatinous mass. The release from a delivery system constituting hydrophilic polymers as taught by the prior art is dependent upon the gastric emptying time and extent of the hydration of the polymer. Polymers being hydrophilic in nature hydrate to form a gel layer on exposure to aqueous fluids, which thereafter slowly dissolves to release the medicament. The rate and extent of hydration of the polymer is dependent on the pH of the media. The rate of release of drug from such a system is primarily dependent on viscosity of the polymer, rate of water imbibition, resultant rate of swelling of polymer, drug dissolution and diffusion from the polymer. The release of medicament is also said to take place by leaching action at or near the surface. However, it is well recognized that the application of such a system to obtain a consistent rate of release of the drug wherein it is regulated by the diffusion of the polymer, is difficult to maintain.

Such a delivery system would be primarily undesirable for drugs that are freely soluble and possess a short elimination half-life. A delivery system based purely on the concept of “bio-erosion” would overcome all of the above-mentioned problems. None of the aforementioned applications describe the use of a bioerodible, water-insoluble polymer, such as ethylcellulose, alone to control the release of metformin irrespective of the environmental pH of the gastrointestinal tract.

### SUMMARY OF THE INVENTION

It is therefore an object of the invention to provide efficacious methods for the development of drug delivery systems of biguanides alone or in combination with other drugs to treat diabetes-associated maladies. Furthermore, in light of the foregoing, the principal object of the present invention is to provide a delivery system for oral administration of biguanide alone or in combination with rosiglitazone or sulfonylureas.

It is an object of the present invention to provide an oral delivery system for the biguanide that provides for a pH-independent controlled release of the drug.

It is a further object of the present invention to provide an oral delivery system for the combination of biguanide and rosiglitazone such that biguanide is provided as a pH-independent controlled release and rosiglitazone is released immediately upon administration.

It is also an object of the present invention to provide an oral delivery system for the combination of biguanide and sulfonylurea such that biguanide is provided in a pH-independent controlled release form.

It is another object of the present invention to provide a bilayered delivery system for oral administration constituting a core of biguanide, and a layer of rosiglitazone or sulfonylurea over the said core.

It is yet another object of the present invention to provide an oral delivery system which comprises a core of biguanide with a water-insoluble polymeric carrier that provides for controlled release of the drug over a prolonged period of time.

It is also an object of this invention to provide a method of use of these compositions for the treatment of diabetes.

These objects are achieved by virtue of the present invention, which relates to an oral delivery system that selectively delivers drugs at an optimal rate and exhibits reproducibility of release rates into the aqueous media at the absorptive regions of the gastrointestinal tract.

In one embodiment of the present invention, an oral delivery system for the treatment of non-insulin dependent diabetes mellitus in humans comprises a pharmaceutically effective amount of metformin with a water-insoluble polymeric carrier that provides for a controlled release independent of environmental pH. A further embodiment of the delivery system of the present invention comprises a core of said metformin and a layer comprising rosiglitazone or sulfonylurea over the core wherein the delivery system provides a pH independent controlled release of metformin over an extended period of time and immediately releases the drug (i.e., rosiglitazone or sulfonylurea) from the layer upon administration.

Another embodiment of the present invention includes a therapeutic system in the form of a bilayered dosage form in a fixed-dose combination for the controlled delivery of biguanide and an immediate release of a rosiglitazone or sulfonylurea to provide a synergistic effect for the treatment of diabetes. Bilayered tablets provide an advantage over core compositions in minimizing the contact between the two biologically active substances and also allow the second drug component to be released immediately whereas the biguanide is released at a controlled rate.

The present invention also includes antidiabetic combinations and processes for the preparation of such compositions comprising combinations of a controlled release biguanide and rosiglitazone for the treatment of non-insulin dependent diabetes mellitus for improving glycemic control. In one embodiment of the invention, a composition comprising an immediate release component and a controlled release biguanide component, all in the same dosage form, can be prepared by the use of a bilayered tablet.

The term “bilayered” as used herein is meant to encompass solid dosage forms such as tablet formulations where there are two separate drug layers, one on top of the other with only one surface in mutual contact. These may be prepared by compressing additional granulation on a previously compressed granulation, or alternatively by feeding previously compressed tablets into a machine and compressing another granulation layer around the preformed tablets. The term “bilayered” also includes formnulations where one drug component is coated onto the second drug component, which may be in the form of, for example, tablets, capsules, granules, pellets or beads.

### BRIEF DESCRIPTION OF THE DRAWINGS

FIG. 1 shows the pH-independent release profile of metformin tablets according to Example 1.

### DETAILED DESCRIPTION

An embodiment of the present invention includes a controlled release delivery system of biguanide that releases the drug independently from environmental pH. Another embodiment of the present invention further includes a second layer comprising rosiglitazone or sulfonylureas that provides for an immediate release of the drug.

The Controlled Release Biguanide

The biguanides that can be used in accordance with the processes and compositions of the present invention include, but are not limited to, metformin, phenformin, buformin and other medicinally active and pharmaceutically acceptable forms from the biguanide class of compounds, including their salts, solvates, hydrates, polymorphs, complexes and such other products. In accordance with the present invention, metformin is a particularly preferred biguanide because of its proven clinical use. Different salts of metformin that could be used in the present invention include hydrochloride, acetate, maleate, fumarate, succinate and other salts, such as the different salts of metformin described in the literature in U.S. Pat. No. 6,031,004, which is incorporated herein by reference in its entirety. The daily effective dose of metformin for the oral treatment of diabetes preferably ranges from 500 mg to 2550 mg, more preferably, the dose is a single dose of 500 mg to 850 mg.

The biguanide is preferably present in an amount from about 45% to about 70% by weight, more preferably from about 50% to about 65% by weight, of the total composition.

According to an embodiment of the present invention, the pharmaceutical composition comprises a bioerodible water-insoluble polymeric carrier for retarding the release of the therapeutic agent. The insoluble polymer would thus control the release of the biguanide based solely on the principle of erosion which is independent of varying pH, as opposed to the pH-dependent swelling behavior of water-soluble hydrophilic cellulosic polymers.

The term “bioerodible polymer” as used herein is intended to mean “those polymers which undergo bioerosion.” Bioerosion includes physical as well as chemical break down of a matrix comprising a polymer. As a result, loss of mass from polymer matrix takes place and the device shrinks with time.

Such polymers swell upon ingress of water following which they erode due to mechanical action or by the dissolving action of the aqueous media on the exposed surface. Such erosion is controlled by the molecular weight of the polymer, hydrophilicity of other excipients, and the like. Specifically, a lower molecular weight polymer would erode faster than a higher molecular weight polymer. These bioerodible polymers ensure a controlled and reproducible release rate of the drug as the system traverses through the variable pH of the gastrointestinal tract.

Examples of polymers which can be used in the present invention include bioerodible polymers exemplified by polylactic acid, polyglycolic acid, polylactones, polyhydroxy butyric acid, polyorthoesters, polyanhydrides, polyamides, their copolymers, and water-insoluble cellulose derivatives. Preferably, the polymer is the water-insoluble bioerodible polymer selected from the group consisting of cellulose and its water-insoluble derivatives. A particularly preferred water-insoluble bioerodible polymer is ethylcellulose of viscosity grades 7, 10, 20 and 100 centipoise and mixtures thereof.

Ethylcellulose may be used in powder form (Ethoce™ 7 cps, Ethoce™ 10 cps, Ethocel™ 20 cps, Ethocel™ 100 cps; Dow Ltd., USA) or as its commercially available aqueous dispersions (Aquacoat™, Surelease™). The composition may comprise ethylcellulose of a specific viscosity grade, or a blend of more than one grade may be employed in accordance with the invention without imposing any restriction on the mode of addition. Alternatively, another embodiment of the invention may include ethylcellulose partly in the powder form and partly as commercially available dispersions. Ethylcellulose may be incorporated along with the drug or it may be applied as a coating, or a combination of the two methods may be used.

The amount of the polymer present in the composition of the invention preferably varies from about 10% to about 40% of the weight of the total composition. More preferably, the amount of polymer present in the composition varies from about 15% to about 35% by total weight of the composition. Most preferably, the amount of polymer present in the composition varies from about 17% to about 32% of the weight of the total composition. Depending upon the desired drug release profile and the presence of other excipients, appropriate adjustments may be made with regard to the amount of polymer present in the composition or with regard to the selection of various grades of polymer, and are within the scope of the present invention.

According to another embodiment of the present invention, the controlled release component of the delivery system further comprises a rate release modifier in conjunction with the water-polymer insoluble polymer, which together regulate the release of the biguanide. The rate of swelling and erosion of the polymer, as mentioned above, varies with the molecular weight of the and hydrophilicity of the other excipients. The molecular weight of the polymer also affects the diffusivity of the swollen polymer. This diffusivity and thus erosion rate may be regulated by the addition of suitable amounts of rate release modifiers. The degree of uniform dispersion of the water-soluble release modifiers in the system ensure quick and regulated erosion that is substantially constant and at a pH-independent rate. The rate release modifiers, which are amenable to controlled release therapy utilizing the novel therapeutic delivery system of the present invention, include any of those suitable for oral administration and that are water-soluble. Examples of such water-soluble agents that may be used in the present invention include, but are not limited to, lactose, dicalcium phosphate, calcium sulphate, mannitol, dextrates, dextrin, dextrose, sucrose, polyethylene glycol, polyvinylpyrrolidone and the like.

The amount of rate release modifier present in the composition of the invention preferably varies from about 2% to about 20%, and more preferably from about 4% to about 18%, by weight of the total weight of the composition.

Other excipients such as binders, bulking agents or fillers, disintegrating agents, glidants, lubricants and others may optionally be used in the preparation of the composition of the invention. Preferably, the binder is selected from the group consisting of hydroxypropyl cellulose, hydroxyethyl cellulose, sodium carboxymethyl cellulose, polyvinylpyrrolidone, alginates, starches, polysaccharides, and/or mixtures thereof. When present, the binder preferably comprises about 3% to about 10% of the total weight of the composition.

Examples of fillers which may be used in the present invention include microcrystalline cellulose, lactose, calcium phosphates, starches, polyols and/or mixtures thereof. When present, the filler preferably comprises about 0% to about 30% of the total weight of the composition.

Suitable disintegrating agents that may be used in the present invention include starch, croscarmellose sodium, sodium starch glycolate, crospovidone, cross-linked carboxymethyl starch, magnesium aluminium silicate, polyacrylin potassium, and the like. When present, the disintegrating agent may be present in an amount from about 1% to about 10% of the total weight of the composition.

Suitable lubricants, glidants and anti-adherents which may be used in the present invention include stearates, stearic acid, colloidal silicon dioxide, talc, soluble lubricants, and/or mixtures thereof. Preferably, the composition of the invention comprises more than one such agent selected from these categories. Most preferably, the composition of the invention comprises a combination of stearates and talc along with colloidal silicon dioxide in an amount from about 0.5% to about 5% by weight of the composition.

Other additives, such as surface active agents, bioavailability modifiers, modifiers for absorption and the like, may be used in the composition of the present invention, and when present, they preferably comprise about 0.5% to 5% of the weight of the composition. Optionally, the composition of the invention may further include buffering agents, complexing agents and the like, without affecting the spirit or scope of the invention.

As would be understood by one of ordinary skill in the art, the additives comprising the composition of the invention may be added in amounts which are required to produce the desired function of the additive in order to manufacture the composition in the form of a suitable dosage form.

The delivery system of the present invention may be prepared in the form of, for example, pellets, beads, granules, tablets or capsules.

The Layer of Another Therapeutic Agent

According to another embodiment of the present invention, the controlled release system of biguanide may also be formulated as a combination product, further comprising a layer of glitazone or sulfonylurea.

In the embodiments of the invention where the second layer of the composition contains a glitazone, a suitable glitazone includes, but is not limited to, troglitazone, pioglitazone, ciglitazone, rosiglitazone and other medicinally active and pharmaceutically acceptable forms from the glitazone class of compounds, including their salts, solvates, hydrates, polymorphs, complexes and such other products. Of these, troglitazone, pioglitazone, and rosiglitazone are preferred compounds because of their proven use and commercial availability. Even more preferred is rosiglitazone because of its low dose (2-8 mg, Avandia™, Smith Kline Beecham) when compared with the relatively higher doses of 15-45 mg for pioglitazone (Actos™, Takeda) and 200-400 mg for troglitazone (Rezulin™, Parke-Davis), thereby rendering rosiglitazone a preferred glitazone for a combination employing a bilayered tablet in accordance with the present invention.

In an embodiment of the invention, the rosiglitazone present in the composition may be released immediately upon oral administration. The low dose and the short half-life of elimination from the human body (3-4 hours, Physician's Desk Reference, 2001, page 2980) of rosiglitazone render it a preferred glitazone for use in a fixed-dose combination of the invention along with other antidiabetics.

In the embodiments of the invention where the second layer of the composition contains a sulfonylurea, a suitable sulfonylurea includes, but is not limited to, glipizide, glimepiride, glibornuride, glyburide, glisoxepide, gliclazide, acetohexamide, chlorpropamide, tolazamide, tolbutamide, and others, and other medicinally active and pharmaceutically acceptable forms from the sulfonylurea class of compounds, including their salts, solvates, hydrates, polymorphs, complexes and such other products. For example, suitable sulfonylureas for use in the present invention are described in U.S. Pat. Nos. 5,674,900 and 4,708,868, both of which are incorporated herein by reference in their entireties. A preferred sulfonylurea for use in the present invention is glipizide. The sulfonylurea as used in accordance with the present invention may be so formulated that it may provide an extended release or an immediate release of the sulfonylurea. For example, in an embodiment of the invention including a combination of controlled release metformin with a sulfonylurea, the sulfonylurea may be so formulated that it can provide an extended release or an immediate release of the sulfonylurea.

Any conventional method may be used for the preparation of the layer of rosiglitazone or sulfonylureas. There may also be incorporated into this component of the delivery system of the present invention, conventional pharmaceutically acceptable excipients known in the art of formulation development, such as diluents, binders, lubricants, and the like. In addition, the excipients described above for the preparation of the biguanide layer could also be used for the preparation of the rosiglitazone or sulfonylurea layer, but with discretion as would be understood by one of ordinary skill in the art. It is to be borne in mind, however, that the conventional pharmaceutical auxiliary additives which might adversely affect the rate of release of the drug are not desirable for use in the present invention. The choice of the different additives to be used for the preparation of the two layers in accordance with the present invention is well within the scope of a person skilled in the art of designing and manufacturing pharmaceutical solid dosage forms.

Another embodiment of the formulation of the invention may contain other various materials that modify the physical form of the dosage unit, for example, as coatings. Thus, an embodiment of the invention includes a core formulation as previously described coated with, for example, water-soluble polymers, sugar, shellac or other enteric coating agents. Preferably, the materials used in preparing these various compositions are pharmaceutically pure and non-toxic in the amounts used.

In an embodiment for the process of making the composition of the present invention, the biguanide is blended with other components of the composition and granulated with a suitable fluid such as water or alcohol, dried, suitably sized and lubricated to obtain granules for the first layer. In a similar fashion, granules for the second layer of, for example, rosiglitazone, are also prepared. The granules for the two layers are then compressed into a bilayered tablet by known methods and equipment. Alternatively, another embodiment for the process of preparing the composition of the present invention includes coating the, for example, rosiglitazone layer onto the, for example, tablet, pellets or granules comprising biguanide. The methods and equipment used for the different unit operations such as blending, granulating, drying, sizing and coating are well known to a person skilled in the art of making pharmaceutical solid dosage forms.

In those embodiments of the present invention wherein the foregoing composition is in the form of spherical pellets or beads, such dosage forms may be produced by the known techniques of extrusion and spheronisation techniques, or techniques based on high shear granulation or fluidized bed techniques, for example. Furthermore, embodiments of single unit pellets can be produced on an industrial scale by using lozenge and troches cutting machines. Alternatively, the components of the compositions of the present invention can be converted to a dosage form, such as a tablet, by direct compression, slugging or roll compaction methods, all of which are known in the art.

Thus, as described herein, the compositions of the invention can be produced without the need for any special equipment or critical process, which is one of the objectives of the invention.

Another embodiment of the composition of the invention offers further flexibility by converting it into particles or pellets which may be coated with the polymer before compressing into tablets or filling into capsules. Other embodiments include other modifications involving coating the tablet with the polymer in order to modify the release of the drug. The composition of the present invention, when presented in the form of solid dosage forms such as tablets, caplets or pills, may be optionally coated with non-functional coatings as are well known in the art, or with coatings which further modify the release of the drug from the said composition of the invention. All such known modifications as may be done and understood by those who are skilled in the art are within the scope of the present invention. For example, one such modification comprises making of the composition of the invention into a layered tablet whereby the composition provides extended release of more than one therapeutic agent, or extended release of one of the therapeutic agents and immediate or delayed release of the other therapeutic agent(s).

It is to be understood, however, that for any particular subject being treated, e.g., a mammal, specific dosage regimens should be adjusted according to the individual need, as would be understood by one of ordinary skill in the art. It is further to be understood that the dosages set forth herein are examples only, and they do not to any extent limit the scope of the present invention.

The formulation of the present invention may be administered orally, for example, with inert diluent or with an edible carrier. For the purpose of oral therapeutic administration, the formulation of the invention may have excipients incorporated therein. The formulation may also contain the following adjuvants: a binder such as microcrystalline cellulose, gum tragacanth or gelatin; an excipient such as starch or lactose; a disintegrating agent such as alginic acid, Primogel™, corn starch, and the like; a lubricant such as magnesium stearate or Sterotex™; a glidant such as colloidal silicon dioxide; and a sweetening agent such as sucrose or saccharin, and/or a flavoring agent such as peppermint, methyl salicylate or orange flavoring.

Metformin is a preferred example of biguanide and is therefore selected as a representative example in conjunction with a glitazone, such as rosiglitazone, or a sulfonylurea for illustrating the formulations of the present invention.

The present invention is illustrated below by reference to the following examples which set forth particularly preferred embodiments. However, it should be understood that these embodiments are illustrative only and are not to be construed as limiting the invention in any way.

### EXAMPLE 1

This example illustrates tablets of metformin exhibiting the controlled release pattern of the present invention. It clearly depicts a pH-independent release profile of the drug. The pharmaceutical composition of this example is given below in Table 1.

TABLE 1

| | % Weight of the |
|---|---|
| Ingredients | composition |
| Metformin HCl | 61.35 |
| Lactose monohydrate | 6.13 |
| Microcrystalline cellulose | 6.13 |
| Ethyl cellulose | 21.47 |
| Magnesium stearate | 1.10 |
| Talc | 1.10 |
| Colloidal silicon dioxide | 0.74 |

In this example, metformin hydrochloride, lactose monohydrate and microcrystalline cellulose were blended and granulated with an aqueous dispersion of ethyl cellulose in a fluidized bed drier. The process was continued until a complete thick coat of ethyl cellulose covered the granules of the drug. The dried granules were sifted through 850 μm mesh (British Standard Sieve (BSS) No. 18). The sized granules were blended with magnesium stearate, talc and colloidal silicon dioxide prior to compression into tablets.

The tablets were then coated with an aqueous dispersion of hydroxy propyl methyl cellulose, titanium dioxide and polyethylene glycol 400 (Opadry Y-1-7000, White) to a weight gain of 1.96%.

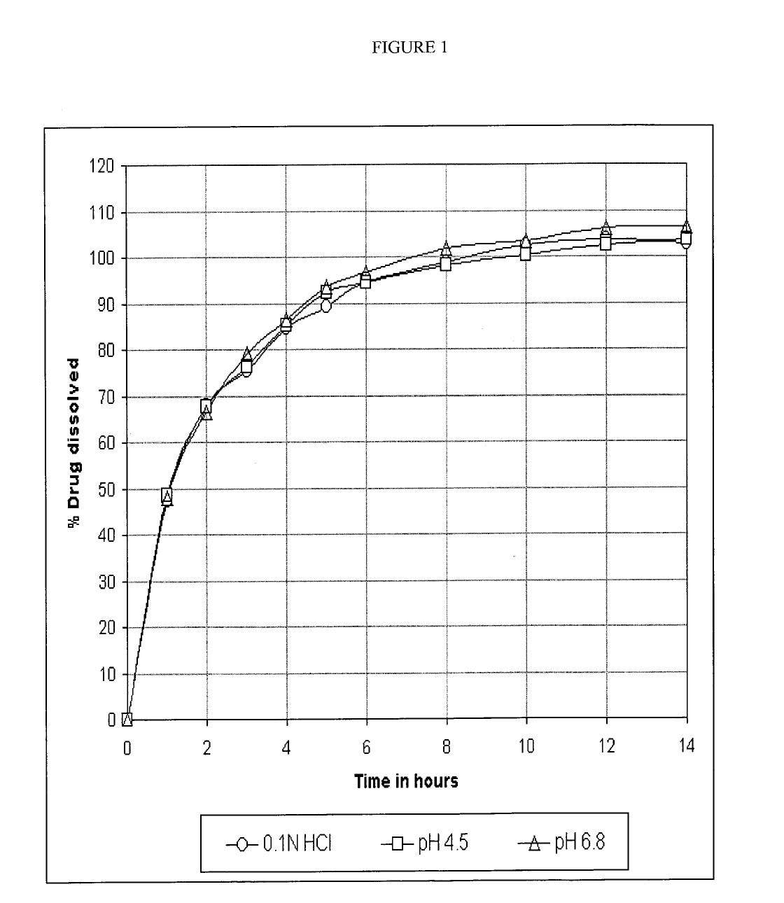

The tablets were characterized for drug release in 900 ml of 0.1N hydrochloric acid and phosphate buffer media of pH 4.5 and pH 6.8. The USP apparatus Type II with paddle speed at 100 rpm was used in this example. The samples of the media were periodically withdrawn and analyzed for drug content. The dissolution results are recorded in Table 2 below, and the profiles are given in FIG. 1.

TABLE 2
Cumulative percent metformin released

| Time | 0.1 N | Phosphate | Phosphate |
|---|---|---|---|
| (Hours) | Hydrochloric acid | buffer, pH 4.5 | buffer, pH 6.8 |
| 1 | 47.4 | 48.5 | 48.1 |
| 2 | 67.9 | 67.6 | 66.4 |
| 3 | 75.3 | 76.2 | 79.2 |
| 4 | 84.5 | 85.3 | 86.6 |
| 5 | 89.1 | 92.4 | 93.5 |
| 6 | 94.6 | 94.6 | 96.6 |
| 8 | 98.7 | 98.3 | 101.9 |
| 10 | 102.6 | 100.4 | 103.5 |
| 12 | 103.7 | 102.6 | 106.2 |
| 14 | 102.8 | 103.6 | 106.5 |

### EXAMPLE 2

This example illustrates the present invention in the form of controlled release tablets of metformin wherein water-insoluble polymer is used both in the form of powder and dispersion to prepare the tablets. The pharmaceutical composition of this example is given below in Table 3.

TABLE 3

| | % Weight of the |
|---|---|
| Ingredients | composition |
| Metformin HCl | 58.0 |
| Dicalcium Phosphate | 11.6 |
| Ethyl cellulose | 29.0 |
| Magnesium stearate | 1.2 |
| Talc | 0.60 |
| Colloidal silicon dioxide | 0.60 |

In this example, a blend of metformin hydrochloride, a partial quantity of ethycellulose, and dicalcium phosphate was prepared in a mixer. A dispersion of the remaining amount of ethylcellulose was prepared in an Isopropyl Alcohol:Acetone mixture to granulate the above blend. The we granules were dried, sifted and blended with magnesium stearate, talc and colloidal silicon dioxide prior to compression into tablets.

The tablets were then characterized for drug release in phosphate buffer media of pH 6.8 as described in Example 1 above, and the dissolution results are recorded in Table 4 below.

TABLE 4

| | Cumulative percent |
|---|---|
| Time (Minutes) | metformin released |
| 60 | 54 |
| 120 | 72 |
| 180 | 84 |
| 240 | 92 |
| 300 | 98 |
| 360 | 101 |

### EXAMPLE 3

This example illustrates the invention in the form of controlled release tablets of metformin wherein higher concentrations of rate release modifier were used to regulate the release profile. The pharmaceutical composition of this example is given below in Table 5.

TABLE 5

| | % Weight of the |
|---|---|
| Ingredients | composition |
| Metformin HCl | 58.7 |
| Dicalcium Phosphate | 17.6 |
| Ethyl cellulose | 14.7 |
| Ethyl cellulose dispersion | 7.3 |
| (Surelease) | |
| Magnesium stearate | 0.60 |
| Talc | 0.60 |
| Colloidal silicon dioxide | 0.60 |

In this example, the tablets were prepared and studied for drug release as described in Example 2. The dissolution results are recorded in Table 6 below.

TABLE 6

| | Cumulative percent |
|---|---|
| Time (Minutes) | metformin released |
| 60 | 59 |
| 120 | 78 |
| 180 | 90 |
| 240 | 98 |
| 300 | 102 |
| 360 | 103 |

### EXAMPLE 4

This example illustrates the present invention in the form of core tablets of metformin coated with a dispersion of rosiglitazone. The pharmaceutical composition of this example is given below in Table 7.

TABLE 7

| | |
|---|---|
| | % Weight of the |
| Ingredients | composition |

Metformin Core:

| | |
|---|---|
| Metformin HCl | 58.33 |
| Lactose monohydrate | 5.83 |
| Microcrystalline cellulose | 5.83 |
| Ethyl cellulose | 25.66 |
| Magnesium stearate | 1.05 |
| Talc | 1.05 |
| Colloidal silicon dioxide | 0.70 |

Rosiglitazone Coat:

| | |
|---|---|
| Dispersion of Hydroxy | 1.24 |
| propyl methyl cellulose, | |
| Titanium dioxide & | |
| Polyethylene Glycol 400 | |
| (Opadry Y-1-7000, White) | |
| Rosiglitazone maleate | 0.31 |

In this example, metformin hydrochloride, lactose monohydrate and microcrystalline cellulose were blended and granulated with an aqueous dispersion of ethyl cellulose in a fluidized bed drier. The process was continued until a complete thick coat of ethyl cellulose covered the granules of the drug. The dried granules were sifted through 850 μm mesh (British Standard Sieve (BSS) NO. 18). The sized granules were blended with magnesium stearate , talc and colloidal silicon dioxide prior to compression into tablets.

Rosiglitazone maleate was dispersed in an aqueous dispersion of Opadry Y-1-7000. This metformin core tablets described above to the weight gain described in Table 7.

The tables were then characterized for drug release in 900 ml of 0.1N hydrochloric acid using USP apparatus Type II with paddle speed at 100 rpm. The samples of the media were periodically withdrawn and analyzed for drug content. The dissolution results are given below in Tables 8 and 9 for metformin and rosiglitazone, respectively.

TABLE 8

| | Cumulative percent |
|---|---|
| Time (Minutes) | metformin released |
| 60 | 45.70 |
| 120 | 62.20 |
| 180 | 73.70 |
| 240 | 80.30 |
| 300 | 86.60 |
| 360 | 89.00 |

TABLE 9

| | Cumulative percent |
|---|---|
| Time (Minutes) | rosiglitazone released |
| 05 | 90.95 |
| 10 | 103.74 |
| 15 | 102.58 |
| 30 | 101.62 |
| 45 | 99.97 |
| 60 | 101.14 |

The results of this example an immediate release profile of rosiglitazone and a controlled release pattern of metformin fraction.

### EXAMPLE 5

This example illustrates the invention in the form of bilayered tablets of metformin and gilipizide. The pharmaceutical composition of this example is given below in Table 10.

TABLE 10

| | |
|---|---|
| | % Weight of the |
| Ingredients | composition |

Metformin Layer:

| | |
|---|---|
| Metformin HCl | 59.24 |
| Lactose monohydrate | 5.92 |
| Microcrystalline cellulose | 5.92 |
| Ethyl cellulose | 26.07 |
| Magnesium stearate | 1.07 |
| Talc | 1.07 |
| Colloidal silicon dioxide | 0.71 |

Glipizide Layer:

| | |
|---|---|
| Glipizide | 5.04 |
| Lactose monohydrate | 50.44 |
| Microcrystalline cellulose | 35.31 |
| Povidone (K-30) | 3.33 |
| Sodium starch glycolate | 3.03 |
| Coloring Agent (FD & C | 0.33 |
| yellow lake) | |
| Magnesium stearate | 1.01 |
| Colloidal silicon dioxide | 0.50 |
| Talc | 1.01 |

In this example, metformin hydrochloride, lactose monohydrate and microcrystalline cellulose were blended and granulated with an aqueous dispersion of ethyl cellulose in a fluidized bed drier. The process was continued until a complete thick coat of ethyl cellulose covered the granules of the drug. The dried granules were sifted through 850 μm mesh (British Standard Sieve (BSS) No. 18). The sized granules were blended with magnesium stearate, talc and colloidal silicon dioxide.

Glipizide, lactose monohydrate, microcrystalline cellulose, coloring agent and sodium starch glycolate were blended and granulated with an aqueous dispersion of povidone. The wet mass was granulated, dried and sifted through 850 μm mesh (British Standard Sieve (BSS) No. 18). The sized granules were blended with magnesium stearate, talc and colloidal silicon dioxide.

The lubricated granules of metformin and glipizide were compressed into bilayer tablets using a rotary compression machine.

The tablets were then characterized for drug release in phosphate buffer media of pH 6.8 as described in Example 1, and the dissolution results of metformin and glipizide are recorded in Tables 11 and 12, respectively.

TABLE 11

| | Cumulative percent |
|---|---|
| Time (Minutes) | metformin released |
| 60 | 46.2 |
| 120 | 62.6 |
| 180 | 72.9 |
| 240 | 81.1 |
| 300 | 87.1 |
| 360 | 91.6 |
| 480 | 98.0 |
| 600 | 101.7 |

TABLE 12

| | Cumulative percent |
|---|---|
| Time (Minutes) | glipizide released |
| 02 | 68.52 |
| 05 | 89.15 |
| 10 | 95.20 |
| 20 | 96.63 |
| 30 | 95.24 |
| 45 | 93.35 |

The results of this example indicate an immediate release profile of glipizide and a controlled release pattern of metformin fraction.

### EXAMPLE 6

This example illustrates the invention in the form of core tablets of metformin coated with a dispersion of glimepiride. The pharmaceutical composition of this invention is given below in Table 13.

TABLE 13

| | |
|---|---|
| | % Weight of the |
| Ingredients | composition |

Metformin Core:

| | |
|---|---|
| Metformin HCl | 54.47 |
| Lactose monohydrate | 10.89 |
| Microcrystalline cellulose | 10.89 |
| Ethyl cellulose | 19.06 |
| Magnesium stearate | 0.98 |
| Talc | 0.98 |
| Colloidal silicon dioxide | 0.65 |

Glimepiride Coat:

| | |
|---|---|
| Dispersion of Hydroxy | 1.85 |
| propyl methyl cellulose, | |
| Titanium dioxide & | |
| Polyethylene Glycol 400 | |
| (Opadry Y-1-7000, White) | |
| Glimepiride | 0.22 |

In this example, the metformin core was prepared as described in Example 4. Glimepiride was dispersed in an aqueous dispersion of Opadry Y-1-7000. This dispersion was coated on the metformin core tablets to the weight gain described in Table 13.

The tablets were then characterized for drug release in phosphate buffer media of pH 6.8 as described in Example 1, and the dissolution results of metformin and glimepiride are recorded in Tables 14 and 15, respectively.

TABLE 14

| | Cumulative percent |
|---|---|
| Time (Minutes) | metformin released |
| 60 | 50.4 |
| 120 | 71.0 |
| 180 | 84.3 |
| 240 | 91.3 |
| 300 | 95.1 |
| 360 | 99.6 |
| 480 | 101.7 |
| 600 | 102.4 |

TABLE 15

| | Cumulative percent |
|---|---|
| Time (Minutes) | glimepiride released |
| 05 | 79.14 |
| 10 | 81.17 |
| 15 | 82.14 |
| 30 | 83.91 |
| 45 | 84.77 |
| 60 | 85.89 |

The results of this example indicate an immediate release profile of glimepiride and a controlled release pattern of metformin fraction.

What is claimed is:

1. An oral delivery system for the treatment of non-insulin dependent diabetes mellitus in humans for the controlled release of a biguanide or pharmaceutically acceptable salt thereof, comprising:
a pharmaceutically effective amount of a biguanide or pharmaceutically acceptable salt of the biguanide; and
a water-insoluble polymeric carrier comprising a water-insoluble polymer;
wherein the delivery system provides a pH-independent, controlled release of the biguanide or pharmaceutically acceptable salt of the biguanide over an extended period of time.

2. The oral delivery system of claim 1, wherein the biguanide comprises metformin.

3. The oral delivery system of claim 1, wherein the biguanide or pharmaceutically acceptable salt of the biguanide comprises about 45% to about 70% by weight of the oral delivery system.

4. The oral delivery system of claim 3, wherein the biguanide or pharmaceutically acceptable salt of the biguanide comprises about 50% to about 65% by weight of the oral delivery system.

5. The oral delivery system of claim 1, wherein the water-insoluble polymer comprises a member selected from the group consisting of polylactic acid, polyglycolic acid, polylactones, polyhydroxy butyric acid, polyorthoesters, polyanhydrides, polyamides, their copolymers, water-insoluble cellulose derivatives, and mixtures thereof.

6. The oral delivery system of claim 5, wherein the water-insoluble polymer comprises ethylcellulose.

7. The oral delivery system of claim 1, wherein the water-insoluble polymer comprises about 10% to about 40% by weight of the oral delivery system.

8. The oral delivery system of claim 7, wherein the water-insoluble polymer comprises about 17% to about 32% by weight of the oral delivery system.

9. The oral delivery system of claim 1, further comprising a rate release modifier.

10. The oral delivery system of claim 9, wherein the rate release modifier comprises a member selected from the group consisting of lactose, calcium sulphate, dicalcium phosphate, mannitol, dextrates, dextrin, dextrose, sucrose, polyethylene glycol, polyvinylpyrrolidone, and mixtures thereof.

11. The oral delivery system of claim 9, wherein the rate release modifier comprises about 2% to about 20% by weight of the oral delivery system.

12. The oral delivery system of claim 1, further comprising a filler, a binder, a disintegrating agent, a glidant, a lubricant, or a mixture thereof.

13. The oral delivery system of claim 1, wherein the oral delivery system is formed into a physical form selected from the group consisting of a pellet, a bead, a granule, a tablet and a capsule.

14. The oral delivery system of claim 13, wherein the physical form is a tablet, and the tablet includes a coating comprising a fast-dissolving film of a water-soluble polymer.

15. The oral delivery system of claim 13, wherein the physical form is a tablet, and the tablet is a chewable tablet including a sweetening agent, a coloring agent, or a flavoring agent.

16. An oral delivery system for the treatment of non-insulin dependent diabetes mellitus in humans for the controlled release of metformin or a pharmaceutically acceptable salt thereof, comprising:
a pharmaceutically effective amount of metformin or pharmaceutically acceptable salt of metformin;
a water-insoluble polymeric carrier; and
a water-soluble rate release modifier;
wherein the delivery system provides a pH-independent, controlled release of the metformin or pharmaceutically acceptable salt of metformin over an extended period of time.

17. An oral delivery system for the treatment of non-insulin dependent diabetes mellitus in humans, comprising:
a core comprising
a pharmaceutically effective amount of metformin or pharmaceutically acceptable salt of metformin, and
a water-insoluble polymeric carrier comprising a water-insoluble polymer; and
a layer comprising rosiglitazone or sulfonylurea over the core;
wherein the delivery system provides a pH-independent, controlled release of the metformin or pharmaceutically acceptable salt of metformin over an extended period of time, and an immediate release of the rosiglitazone or sulfonylurea from the layer upon administration.

18. The oral delivery system of claim 17, wherein the metformin or pharmaceutically acceptable salt of metformin comprises about 45% to about 70% by weight of the core.

19. The oral delivery system of claim 18, wherein the metformin or pharmaceutically acceptable salt of metformin comprises about 50% to about 65% by weight of the core.

20. The oral delivery system of claim 17, wherein the water-insoluble polymer comprises a member selected from the group consisting of polylactic acid, polyglycolic acid, polylactones, polyhydroxy butyric acid, polyorthoesters, polyanhydrides, polyamides, their copolymers, water-insoluble cellulose derivatives, and mixtures thereof

21. The oral delivery system of claim 20, wherein the water-insoluble polymer comprises ethylcellulose.

22. The oral delivery system of claim 17, wherein the water-insoluble polymer comprises about 10% to about 40% by weight of the core.

23. The oral delivery system of claim 22, wherein the water-insoluble polymer comprises about 17% to about 32% by weight of the core.

24. The oral delivery system of claim 17, wherein the core further comprises a rate release modifier.

25. The oral delivery system of claim 24, wherein the rate release modifier comprises a member selected from the group consisting of lactose, calcium sulphate, dicalcium phosphate, mannitol, dextrates, dextrin, dextrose, sucrose, polyethylene glycol, polyvinylpyrrolidone, and mixtures thereof.

26. The oral delivery system of claim 24, wherein the rate release modifier comprises about 2% to about 20% by weight of the core.

27. The oral delivery system of claim 17, wherein the sulfonylurea comprises a member selected from the group consisting of glipizide, glimepiride, gliboruride, glyburide, glisoxepide, gliclazide, acetohexamide, chlorpropamide, tolazamide, tolbutamide and pharmaceutically acceptable salts thereof.

28. The oral delivery system of claim 27, wherein the sulfonylurea comprises glipizide.

29. The oral delivery system of claim 17, wherein the layer is compressed or coated onto the core.

30. The oral delivery system of claim 17, further comprising a filler, a binder, a disintegrating agent, a glidant, a lubricant, or a mixture thereof.

31. The oral delivery system of claim 17, wherein the oral delivery system is formed into a physical form selected from the group consisting of a pellet, a bead, a granule, a tablet and a capsule.

32. The oral delivery system of claim 31, wherein the physical form is a tablet, and the tablet includes a coating comprising a fast-dissolving film of a water-soluble polymer.

33. The oral delivery system of claim 31, wherein the physical form is a tablet, and the tablet is a chewable tablet including a sweetening agent, a coloring agent, or a flavoring agent.

34. A controlled release composition comprising:
a pharmaceutically effective amount of a biguanide or pharmaceutically acceptable salt of the biguanide; and
a water-insoluble polymeric carrier comprising a water-insoluble polymer;
wherein the composition provides a pH-independent, controlled release of the biguanide or pharmaceutically acceptable salt of the biguanide over an extended period of time.

35. The composition of claim 34, wherein the biguanide or pharmaceutically acceptable salt of the biguanide comprises about 45% to about 70% by weight of the composition.

36. The composition of claim 34, wherein the biguanide comprises metformin and the water-insoluble polymer comprises ethylcellulose.

37. The composition of claim 34, further comprising a rate release modifier selected from the group consisting of lactose, calcium sulphate, dicalcium phosphate, mannitol, dextrates, dextrin, dextrose, sucrose, polyethylene glycol, polyvinylpyrrolidone, and mixtures thereof.

38. A composition comprising:
a core comprising
a pharmaceutically effective amount of metformin or pharmaceutically acceptable salt of metformin, and
a water-insoluble polymeric carrier comprising a water-insoluble polymer; and
a layer comprising rosiglitazone or sulfonylurea over the core;
wherein the composition provides a pH-independent, controlled release of the metformin or pharmaceutically acceptable salt of metformin over an extended period of time, and an immediate release of the rosiglitazone or sulfonylurea from the layer upon administration.

39. The composition of claim 38, wherein the metformin or pharmaceutically acceptable salt of metformin comprises about 45% to about 70% by weight of the composition.

40. The composition of claim 38, wherein the water-insoluble polymer comprises ethylcellulose.

41. The composition of claim 38, wherein the core further comprises a rate release modifier selected from the group consisting of lactose, calcium sulphate, dicalcium phosphate, mannitol, dextrates, dextrin, dextrose, sucrose, polyethylene glycol, polyvinylpyrrolidone, and mixtures thereof.

42. A process for preparing a controlled release composition for the treatment of non-insulin dependent diabetes mellitus in humans, the process comprising:
mixing a pharmaceutically effective amount of a biguanide or pharmaceutically acceptable salt of the biguanide with a water-insoluble polymeric carrier comprising a water-insoluble polymer;
wherein the delivery system provides a pH-independent, controlled release of the biguanide or pharmaceutically acceptable salt of the biguanide over an extended period of time.

43. A process for preparing a composition for the treatment of non-insulin dependent diabetes mellitus in humans, the process comprising:
mixing a pharmaceutically effective amount of a biguanide or pharmaceutically acceptable salt of the biguanide with a water-insoluble polymeric carrier comprising a water-insoluble polymer to form a core; and
forming a layer comprising rosiglitazone or sulfonylurea over the core;
wherein the composition provides a pH-independent, controlled release of the biguanide or pharmaceutically acceptable salt of the biguanide over an extended period of time, and an immediate release of the rosiglitazone or sulfonylurea from the layer upon administration.

44. The process of claim 43, wherein the biguanide comprises metformin and the water-insoluble polymer comprises ethylcellulose.
